(19) United States
(12) Patent Application Publication
(10) Pub. No.: US 2025/0304993 A2
(43) Pub. Date: Oct. 2, 2025

(54) REGULATION OF PROTEIN FUNCTION BY INSERTION OF INTERACTION PEPTIDES

(71) Applicant: KEMIJSKI INSTITUT NATIONAL INSTITUTE OF CHEMISTRY, Ljubljana (SI)

(72) Inventors: Roman JERALA, Ljubljana (SI); Tjaša PLAPER, Šmarješke toplice (SI); Estera MERLJAK ZUPANČIČ, Ljubljana (SI); Tina FINK, Zabnica (SI)

(73) Assignee: KEMIJSKI INSTITUT NATIONAL INSTITUTE OF CHEMISTRY, Ljubljana (SI)

(21) Appl. No.: 18/720,602

(22) PCT Filed: Dec. 13, 2022
(86) PCT No.: PCT/EP2022/085613

§ 371 Date: Jun. 14, 2024

(30) Foreign Application Priority Data
Dec. 15, 2021 (LU) 501018

(51) Int. Cl. C12N 15/85

(52) U.S. Cl. CPC C12N 15/85

(57) ABSTRACT

The invention refers to the regulation of function of proteins through insertion of a peptide into the selected protein and its interaction with a regulatory peptide that interacts with the inserted peptide. The invention can be used to activate or inactivate the function of different selected proteins and therefore to regulate their properties and processes, useful for pharmacological, therapeutic, diagnostic, sensing, biotechnological and other industrial applications.

### CROSS REFERENCE TO RELATED APPLICATION(S)

This application is a 35 U.S.C. § 371 National Stage of International Patent Application No. PCT/EP2022/085613, filed Dec. 13, 2022, which claims priority and the benefit of Luxembourg Patent Application No. LU501018, filed Dec. 15, 2021, the disclosures of which are incorporated herein by reference in their entirety.

### FIELD OF THE INVENTION

The invention refers to the regulation of function of proteins through insertion of a peptide into the selected protein and its interaction with a regulatory peptide that interacts with the inserted peptide. The method involves insertion of a non-structured peptide, preferably into the solvent exposed loop of the target protein, wherein the target protein retains its function. When a regulatory peptide is added that binds to the inserted peptide, it forms an elongated defined structure that disrupts the functional site of the target protein by an allosteric effect and disrupts the function of said selected protein. Genetic fusion of an inhibitory peptide to the selected protein, wherein said inhibitory peptide interacts with an inserted peptide within the same polypeptide chain results in inhibition of the function of the selected protein. Said selected protein can be reactivated by the addition of a regulatory peptide that binds with higher affinity to the fused inhibitory peptide or by proteolytic cleavage of the linker peptide between the said protein and an inhibitory peptide. The invention can be used to activate or inactivate the function of different selected proteins and therefore to regulate their properties and processes such as enzymatic activity, recognition and binding to other molecules, signaling and cellular localization, useful for pharmacological, therapeutic, diagnostic, sensing, biotechnological and other industrial applications.

### STATE OF THE ART

Regulation of the biological activity of proteins through interactions with other molecules is a cornerstone of complex biological systems and is achieved by a variety of mechanisms, from ligand binding to proteolysis and many others. In most cases, the posttranslational regulation of protein activity is uniquely adapted to each protein and is difficult to translate to other proteins. Therefore, it would be desirable to have a widely applicable principle of protein function regulation that could be introduced into diverse natural and engineered proteins that could be used for therapeutic, biotechnological and other purposes. Several engineering principles have already been introduced, such as combinations with degrons (CHOMP, Gao et al., 2018), fusion with coiled-coil and proteolysis domains (SPOCK, Fink et al., 2019)), and engineering of interaction domains modeled to introduce alternative arrangements of the secondary structural elements (LOCKR, Langan et al., 2019). However, these systems have limited applicability and require extensive remodeling of the target protein.

Another method of regulating protein activity is based on split proteins, in which protein activity is reconstituted by the proximity of the two split protein chains, which can be triggered by fusion to form interaction domains. It would be sufficient and even advantageous if the two subdomains would remain on the same polypeptide chain and be only slightly displaced to disrupt the function of the protein.

Allostery is a principle of modulation of protein function through conformational changes that occur when a regulator binds at a site remote from the primary functional site. Allosterically engineered proteins have been designed by introducing folded protein domains or domains that compete with the folding of the target protein (Dagliyan et al., 2013, 2019; Ha & Loh, 2012; Ostermeier, n.d.). There are a couple of designed allosterically regulated proteins, that have been designed individually for each protein, based on the introduction of protein domains that are regulated by binding of a ligand that induces complex formation (Dagliyan et al., 2017) or by alternate frame folding (Mitrea et al., 2010), which however need to be extensively tuned for each target protein separately and have not been used for diverse proteins. A method of regulation of the function of al or at least large majority of proteins through the addition of short peptides or small molecules could have important biomedical and biotechnological use. Further it would be of great biotechnological and biomedical use if the invention could provide a way to convert the inactive into an active functional protein by the addition of a peptide or activation of a protease, which can be activated by a small molecule.

### SUMMARY OF THE INVENTION

The present invention refers to the regulation of the function of selected target proteins by insertion of a non-structured peptide into an appropriate position that maintains the particular function of the target protein. Upon addition of a regulatory peptide that binds to the inserted peptide, a coiled-coil dimer is formed between the regulatory peptide and an inserted peptide, changing its conformation from a random structure to the helical structure, which increases the distance between the termini of the inserted peptide. This locally disrupts the structure of the target protein and inhibits its function. The invention can be used to activate or inactivate the function, structure and properties of a target protein and can be used to regulate processes such as enzymatic activity, recognition of other molecules, signaling, binding to other molecules, cellular localization, optical and other properties, which are useful for pharmacological, therapeutic, diagnostic, sensing, biotechnological and industrial applications.

In the present invention, the appropriate target protein site is defined by testing the function of the target protein with a peptide inserted at different, typically solvent-exposed, loop sites that do not participate directly in protein function. The position of the insertion within the selected target protein is selected in such way that the target protein with an insert peptide retains the selected function. In the presence of an interaction peptide, the target peptide forms a coiled-coil dimer with a peptide inserted into the target protein loop, which impairs the function of the selected target protein.

The present invention refers to the method of activation of a target protein function, wherein the target protein is initially inhibited through insertion of a peptide as described above and an additional fusion of an inhibitory peptide that interacts weakly with an inserted peptide and forms a coiled coil dimer with a peptide inserted into the loop of a target protein, wherein the inhibitory peptide is genetically fused to the target protein via a flexible peptide linker. Said target protein can be activated by the addition of a regulatory peptide that strongly interacts with the inhibitory peptide or through a protease, that cleaves the linker between the target protein and the inhibitory peptide. The present invention can be further implemented to regulate the function of the target protein by a protease whose activity can be regulated through small molecules or though the biological processes, such as a protease specific for the cell type, cell state or a microbial protease, for example viral protease.

The present invention can be further implemented to combine several input signals to implement logic functions that activate or inhibit the function of the target protein, depending on the combination of coiled-coil forming peptides and proteases.

This invention refers to activation or inhibition of the biological function or biological activity such as catalytic activity or binding or other function of various target proteins such as enzymes, such as luciferase, hydrolases, kinases or proteases, proteins that constitute signaling pathways, such as protein kinases, signaling mediators that recruits other proteins to the signaling complex. Further it refers to proteins, which bind specific nucleic acid sequences or that act as transcriptional regulators, and proteins that can be detected and act as reporters, such as fluorescent proteins or luciferases and further it refers to other molecule-recognizing domains such as antibodies and their domains or to structural proteins.

The disclosed invention refers to functional proteins whose activity can be inhibited or activated by the addition or presence of a chemical or biological signal that affects the formation of the dimer involving inserted peptide in the loop of the target protein.

The disclosed invention refers to the method of regulating the activity of proteins and said proteins and nucleic acids encoding them and cells producing said proteins.

In the particular embodiment, the disclosed invention refers to the activation or inhibition of the biological and biochemical function of proteins luciferase, beta-galactosidase, IRAK-1 kinase, MyD88, Lyn kinase, transcription activator-like effector and its implementation as DNA binding domains, Cas9 gRNA-dependent DNA nuclease or specific DNA binding domain, fluorescent proteins, antibody variable domain (Fv), whose activity can be regulated through interactions with a peptide that forms a coiled-coil heterodimer with a peptide inserted into the loop of the target proteins.

In the particular embodiments, the cells such as the mammalian or eukaryotic or bacterial cells, exhibit the function of target proteins depending on the applied signal, which triggers formation or reverses formation of a coiled-coil dimer within the target protein loop, that results in the modification of the biological properties, or cells that produce the modified target proteins, which can be used for medical, biotechnological or other application of cell function.

Moreover, the present invention refers to nucleotide sequences comprising coding sequences for polypeptides described above according to the invention, optionally incorporated in a delivery vector such as a plasmid, a linear or circular nucleic acid or a virus or inserted into the genome of cells. In a further aspect, the present invention refers to a protein comprising the polypeptide according to the invention.

### BRIEF DESCRIPTION OF THE FIGURES

FIG. 1: The principle of Inserted peptide structure allosteric regulation (INSRTR) of target protein function.

Scheme of the principle of the invention; where insertion of an unstructured inserted peptide (INS) into the loop of the target protein maintains the protein function, while the addition of a coiled-coil regulatory peptide (REG) triggers formation of a coiled-coil dimer, which extends the inserted peptide and inactivates the target protein function.

FIG. 2: The influence of linker length on the maintenance of the initial function of target protein.

Peptide N8 was genetically inserted at amino acid K493 of firefly luciferase with different lengths of linkers (fLuc_N8, fLuc_5gs:N8:5gs or fLuc_10gs:N8:10gs) at each side of the inserted peptide sequence. White bar presents the activity of luciferase by insertion of an inserted peptide and shaded show the remaining luciferase activity by coexpression of the N7 peptide by transfection of HEK293T cells by different amount of the plasmid coding for N7 regulatory peptide. Linker regions allow for the additional flexibility around the peptide which maintains the firefly luciferase activity as well as the level of inhibition with the addition of peptide pair. 5gs linker flanking inserted peptide N8 demonstrated the best ratio between ON and OFF state.

FIG. 3: CC pair affinity determines the level of inhibition in co-expression.

Demonstration of inhibition level of fLuc_5gs:N8:5gs with REG peptides with different affinity. We used REG peptides which had complementary electrostatic motives to N8 (INS), but different helical propensities, to demonstrate how different CC stability facilitates different levels of regulation. N7 with highest affinity to N8, inhibits luciferase activity most efficiently.

Inserted peptide N8 was genetically fused into the firefly luciferarase. It can bind several different N7 variants designed in a range of binding affinities with P7SN weakest affinity with N8 and N7 having strongest affinity for N8. Co-transfection in HEK293T cells demonstrates that the degree of inhibition of luciferase with inserted N8 peptide depends on the binding affinity of the co-expressed N7 peptide variant.

FIG. 4: Design of inverted INSRTR (ON switch).

Scheme of the design of inverted INSRTR (ON switch). ON switch was designed based on affinity of CC dimer-forming peptides. CC-peptide P7 (INS) with flanking 5gs linkers was genetically inserted into firefly luciferase. Lower affinity CC-forming peptide (INH) was genetically fused on the C-terminal via linker peptide (inhibitory peptide) and into the loop (inserted peptide) of target protein. Due to high local concentration, the inhibitory and inserted peptide form a coiled coil, which results in inactive target protein (OFF state). The addition of a regulatory peptide (REG), with high affinity for CC dimer formation with inhibitory peptide, removes the INH peptide which results in formation of inhibitory and regulatory peptide CC heterodimer and releases the inserted peptide (INS) and target protein regains its activity (ON state).

FIG. 5: Triggers of INSRTR and kinetics in mammalian cell activation.

N7 peptide and release of inhibition can be delivered through several methods in the inverted INSRTR system; A) Delivery of REG N7 peptide via coexpression, regulated by transcription; B) Delivery of synthetic REG N7 peptide via liposomal agent DOTAP, externally added peptide with transfection reagent. In addition to different delivery systems, we demonstrated how the addition of a binding N7 REG peptide, regardless of type of delivery regulates luciferase activity. Orthogonal P4 REG peptide that does not bind to the inserted peptide has no effect. C) Scheme of chemically activated protease regulation of INSRTR; protease PPVp that cleaves the linker peptide between the inhibitory peptide and target protein is activated by a chemical signal, in this case Rapa (rapamycin) that leads to the interaction between FKBP and FRB that are fused to the split PPVp protease.

FIG. 6: Design of INSRTR building modules for allosteric logic circuits.

Scheme of the design principle of building modules for allosteric logic circuits based on allosteric regulation. Target protein with inserted peptide (INS) was genetically fused to the inhibitory peptide (INH) by flexible linker (link) with inserted protease cleavage site. Addition of protease results in proteolytic cleavage at the protease cleavage site, releasing the inhibitory peptide from target protein.

FIG. 7: Scheme for the construction of logic functions based on a combination of proteases or chemical signals affecting the function of a target protein

FIG. 8: Construction of logic gates based on the intramolecular interacting CC segments.

HEK293T cells were transfected with plasmids coding for allosterically regulated luciferase constructs as schematically represented in FIG. 7. A) Implementation of INSRTR logic gates. Different combinations of proteases were used as input signals and the output of logic functions were represented as the luciferase activity. Addition of a protease leads to cleavage of the linker between the luciferase and the inhibitory peptide (INH), resulting in either OFF or ON output. B) Time dependence of the response of luciferase in the system corresponding to the B logic function. Exogenous regulation of rapamycin and abscisic acid mediated reconstitution of PPV and SbMV proteases resulted in fast kinetics activation of inverted INSRTR system

FIG. 9: Characterization of β-galactosidase INSRTR variants.

Peptide N8 was inserted into β-galactosidase on various positions. A) β-galactosidase an exoglycosidase, which hydrolyzes the β-glycosidic bond formed between a galactose and its organic moiety. Activity of all designed β-galactosidase INSRTR versions in the absence or presence of 100 ng of plasmid encoding for regulatory peptide mCherry_N7. B) Insertion position of INS peptide N8 shown here is A239. Inhibition of β-galactosidase_A239 INSRTR variant by titration of co-transfected plasmid encoding regulatory peptide N7.

FIG. 10: Characterization of TEVp INSRTR variants.

Peptide P7 was inserted into TEVp at various positions. A) Activity of all designed TEVp INSRTR versions in the absence or presence of 50 ng of plasmid coding for a regulatory peptide mCherry_N8, TEV (Tobacco-etch virus) protease is a highly sequence-specific cysteine protease. B) Insertion position of INS peptide P7 with flanking 5gs linkers shown here is 177. Inhibition of TEVp_I77 INSRTR variant by titration of co-transfected plasmid encoding peptide N8, C) Activation of TEVp_I77 INSRTR variant (ON switch) with inserted peptide P7, fused to inhibitory peptide by titration of co-transfected plasmid encoding regulatory peptide N7. Autoinhibited (TEVp) with INS peptide P7, INH peptide N8.

FIG. 11: Characterization of Lck INSRTR variants.

Peptide P7 was inserted into Lck at various positions. A) Activity of designed Lck INSRTR variants in the absence or presence of 50 ng plasmid encoding for the regulatory peptide mCherry_N8, Lck kinase, a member of the tyrosine kinases in lymphocytes. B) The insertion position of INS peptide P7 with flanking 5gs linkers shown here is N266. Inhibition of INSRTR variant Lck_N266 by titration of a co-transfected plasmid encoding regulatory peptide N8.

FIG. 12: Characterization of mIRAK-1 INSRTR variants.

Peptide N8 was inserted into mIRAK-1 at various positions. A) Activity of designed mIRAK-1 INSRTR variants in the absence or presence of 90 ng of plasmid encoding for regulatory peptide mCherry_N7; the interleukin-1 receptor (IL-1R) associated kinase 1 (mIRAK1) is a member of serine/threonine-protein kinase. B) Insertion position of INS peptide N8 with flanking 5gs linkers shown here is F212. Inhibition of mIRAK-1_F212 INSRTR variant by titration of co-transfected plasmid encoding regulatory peptide N7.

FIG. 13: Characterization of mMyD88 INSRTR variants.

Peptide N8 was inserted into mMyD88 at various positions. A) Activity of designed mMyD88 INSRTR variants in the absence or presence of 90 ng of plasmid coding for the regulatory peptide mCherry_N7. MyD88 is involved in the innate immune response as adaptor protein in Toll-like receptor and IL-1 receptor signaling pathway. B) Insertion position of INS peptide N8 with flanking 5gs linkers shown here is N170. Inhibition of mMyD88_N170 INSRTR variant by titration of co-transfected plasmid encoding regulatory peptide N7.

FIG. 14: Characterization of TALE-A INSRTR variants.

Transcription activator-like effectors (TALEs) is a DNA binding protein that in fusion with VP16 activator domain acts as a transcription activator. Peptide N6 was inserted into TALE-A at T496 position. Peptide N8 was fused at the C-terminal of TALE-A protein. Activation domain VP16 was brought to the DNA binding site by N8-N7 CC dimer formation. A) Insertion position of INS peptide N6 shown here is T496. Inhibition of TALE-A_T496 INSRTR variant by titration of co-transfected plasmid encoding regulatory peptide N5 with NLS signal. B) Activation of TALE-A_T496 INSRTR variant with inserted peptide N6, fused inhibitory peptide N5 by titration of co-transfected plasmid encoding a regulatory peptide N6 with NLS signal. Autoinhibited TALE with INS peptide N6, INH peptide N5.

FIG. 15: Characterization of dCas INSRTR variants.

Peptide N8 was inserted into dCas9 at various positions. A) Activity of designed dCas9 INSRTR versions in the absence or presence of 90 ng of plasmid encoding for regulatory peptide mCherry_N7. Nuclease-dead Cas9 (dCas9) is a catalytically inactive mutant of Cas9 protein. B) Inhibition of dCas9_R535 INSRTR variant by titration of co-transfected plasmid encoding peptide N7 with NLS signal. Insertion position of INS peptide N8 with flanking 5gs linkers shown here is R535. C) Activation of dCas9_R535 INSRTR variant with inserted peptide P7, fused inhibitory peptide N8 by titration of co-transfected plasmid encoding peptide N7 with NLS signal. Autoinhibited dCas with INS peptide P7, INH peptide N8.

FIG. 16: Characterization of scFv-CD19 INSRTR variants.

CAR-T receptor molecule; Single-chain variable fragment of CD19 antibody (scFv-CD19). Peptide P7 was inserted into scFV CD19 CAR at S196 position.

FIG. 17: Characterization of ngGFP INSRTR variants

HEK293T cells were transfected with plasmids coding for fluorescent protein Neon green (ngGFP) with INSRTR strategy and Regulatory peptide (REG) fused to fluorescent protein mCherry. A) Peptide P7 was inserted at position C143. Inhibition of ngGFP_C143 green fluorescent protein by co-transfection with mCherry fused N8. Neon green is a green fluorescent protein. Insertion position of INS peptide P7 with flanking 5gs linkers shown here is C143. B) Activation of ngGFP_C143 INSRTR variant with inserted peptide P7, fused inhibitory peptide N8 by co-transfection with mCherry fused to N7. Autoinhibited ngGFP with INS peptide P7, INH peptide N8.

### Definitions

The term “target protein” refers to any selected protein whose function is modified by insertion of a (coiled-coil dimer forming) peptide into one of its solvent exposed loops.

The term “inserted peptide” refers to a peptide that is inserted into the loop of the target protein, wherein the peptide by itself is preferentially not folded but has tendency to form a complex, preferentially a coiled-coil dimer upon interaction with a regulatory or inhibitory peptide.

The term “regulatory peptide” refers to a peptide that is added and interacts with an inserted peptide in the loop of the target protein forming a structured complex such as the coiled-coil dimer or interacts strongly with an inhibitory peptide.

The term “inhibitory peptide” refers to a peptide that interacts, preferentially forming a coiled-coil dimer, with an inserted peptide wherein the inhibitory peptide is genetically fused to the target protein at its N- or C-terminal ends or both ends and is connected to a target protein through a flexible peptide linker, preferably consisting of flexible small hydrophilic amino acid residues of sufficient length, typically 10-30 amino acid residues, long enough to enable the inhibitory peptide to bind to the inserted peptide within the same molecule.

The term “coiled-coil dimer” refers to the polypeptide motif composed of two peptides, either as individual molecules or as segments within the protein, that have the propensity to interact specifically with each other to form a dimer of helices that are typically wound each around other to form an elongated superhelix. The term “coiled-coil dimer” as used herein, unless explicitly specified, refers to two different peptides forming a heterodimer.

The term “peptide”, as used herein, refers to the polymeric form of amino acids, shorter than protein, typically shorter than 60 amino acid residues, which is used to interact with another peptide or to connect functional segments of proteins or peptides or to have some other biological or chemical function.

The term “protein”, as used herein, refers to the polymeric form of amino acids of any length, which expresses any function, for instance localizing to a specific location, localizing to specific DNA sequence, facilitating and triggering chemical reactions, transcription regulation, structural function, and biological recognition.

The term “target protein” as used herein refers to the protein whose function is modified by modifications such as the insertion of a peptide, genetic fusion with the inhibitory peptide ad addition of a regulatory peptide or addition of a small molecule that affects the activity of a protease or other proteins.

The term “function” of a target protein refers to the property of the protein that is characteristic for each protein and includes the catalytic activity, tertiary structure, dynamics, stability, interactions with other molecules, physicochemical properties and biological activity either in vitro or within cells or organisms.

The term “protein domain”, as used herein, refers to a folding functional unit of a protein. For example, a part of a protein that can be folded and expressed independently of the entire protein and is typically composed of one or more secondary structural elements, such as alpha helices or beta strands.

The term “antibody” as used herein refers to the protein that is able to bind to other molecules due to the amino acid sequence and conformation of the loops of the proteins that contribute to interactions whereas this protein belongs to the immunoglobulin family and refers also to the antibody fragments comprising variable antibody domain in two chains or connected into a single chain or nanobodies and its molecule binding fragments, which are composed of a single heavy chain.

The term “enzyme” as used herein refers to the protein whose function is to catalyze a reaction so that it occurs faster, with higher efficiency under the physiological conditions.

The term “allostery” or “allosteric” refers to the effect that interaction of an effector molecule with a target protein has on the properties of the protein, such as its activity or binding, while the effector allosteric binding to site is not the same site, which is involved in protein function.

The term “INSRTR” used herein refers to the technique of affecting the function or properties of the target protein through the addition of a regulatory peptide that interacts with either inserted or inhibitory peptide genetically incorporated into the target protein.

The term “ligand”, used herein, refers to any small molecule with low molecular weight (<5000 Da and preferably <900 Daltons). The ligands include but are not limited to for example lipids, monosaccharide, second messengers, hormones, inhibitors, other natural products and metabolites, as well as drugs and other synthetic small molecules.

The term “sensor”, used herein refers to a molecule or molecular complex where the presence of a selected ligand triggers the generation of measurable output signal as e.g. emitted light, fluorescence, electric current, or other chemical or physical signal or change of physicochemical property.

The term “genetic fusion”, as used herein refers to the insertion of DNA coding for the inserted peptide into the DNA coding region of the target protein, wherein the resulting DNA codes for the protein with an inserted peptide, which can be fused to the amino- or carboxy terminus of said protein or inside the protein polypeptide sequence. The single chain polypeptide comprises polypeptide of two or more constituents that are consecutive or between them are short linker polypeptides that prevent steric overlap, typically comprising 1-10 small polar flexible amino acid residues, typically glycine or serine or similar small hydrophilic amino acid residues.

The term “cell”, used herein, refers to a eukaryotic or prokaryotic cell, a cellular or multicellular organism (cell line) cultured as a single cell entity that has been used as a recipient of nucleic acids and includes the daughter cells of the original cell that has been genetically modified by the inclusion of nucleic acids. The term refers primarily to cells of higher developed eukaryotic organisms, preferably vertebrates, preferably mammals. This invention relies also on non-vertebrate cells, preferably plant cells.

The term “cells” also refers to human or animal primary cells or cell lines. Naturally, the descendants of one cell are not necessarily completely identical to the parents in morphological form and its DNA complement, due to the consequences of natural, random or planned mutations. A “genetically modified host cell” (also “recombinant host cell”) is a host cell into which the nucleic acid has been introduced. The eukaryotic genetically modified host cell is formed in such a way that a suitable nucleic acid or recombinant nucleic acid is introduced into the appropriate eukaryotic host cell. The invention hereafter includes host cells and organisms that contain a nucleic acid according to the invention (transient or stable) bearing the operon record according to the invention. Suitable host cells are known in the field and include eukaryotic cells. It is known that proteins can be expressed in cells of the following organisms: human, rodent, cattle, pork, poultry, rabbits and the like. Host cells may include cultured cell lines of primary or immortalized cell lines.

The term “recombinant”, as used herein, means that a particular nucleic acid (DNA or RNA) is a product of various combinations of cloning, restriction and/or ligation or chemical synthesis leading to a construct having structurally coding or non-coding sequences different from endogenous nucleic acids in a natural host system.

The term “nucleic acid”, used herein, refers to a polymeric form of nucleotides (ribonucleotides or deoxyribonucleotides) of any length and is not limited to single, double or higher chains of DNA or RNA, genomic DNA, cDNA, DNA-RNA hybrids, or polymers with a phosphorothioate polymer backbone made from purine and pyrimidine bases or other natural, chemical or biochemically modified, synthetic or derived nucleotide bases.

The term “DNA-binding domain”, used herein, refers to any protein domain with the ability to bind a DNA molecule. The DNA-binding protein could be of natural origin or artificially designed whole protein or only a segment with characteristic to bind to nucleic acid in sequence specific manner.

The term “effector domain”, in the description refers to any protein domain with a specific function, for example, but not limited to nuclease domains, transcriptional activation domains, and chromatin silencing domains.

The insertion of the vectors into the host cells is carried out by conventional methods known from the field of science, and the methods relate to transformation or transfection and include e.g.: chemically induced insertion, electroporation, micro-injection, DNA lipofection, cellular sonication, gene bombardment, viral DNA input, as well as other methods. The entry of DNA may be of transient or stable. Transient refers to the insertion of a DNA with a vector that does not incorporate the DNA of the invention into the cell genome. A stable insertion is achieved by incorporating DNA of the invention into the host genome. The insertion of the DNA of the invention, in particular for the preparation of a host organism having stably incorporated a nucleic acid, e.g. a DNA, of the invention, can be screened by the presence of markers. The DNA sequence for markers refers to resistance to antibiotics or chemicals and may be included on a DNA vector of the invention or on a separate vector.

### DETAILED DESCRIPTION OF THE INVENTION

The present invention refers to the regulation of the function of selected target proteins by insertion of a peptide, preferably in the range between 20 and 50 amino acid residues (e.g. 30-40 amino acid residues), into an appropriate position that maintains the particular function of the target protein. Upon addition of a regulatory peptide that binds to the inserted peptide, a coiled-coil dimer is formed between the regulatory peptide and the inserted peptide, changing its conformation from a random structure to a helical structure, which increases the distance between the termini of the inserted peptide. This locally disrupts the structure of the target protein and inhibits its function. The invention can be used to activate or inactivate function, structure and other properties of a target protein and can be used to regulate processes such as enzymatic activity, recognition of other molecules, signaling, binding to other molecules, cellular localization, optical and other properties, which are useful for pharmacological, therapeutic, diagnostic, sensing, biotechnological and industrial applications.

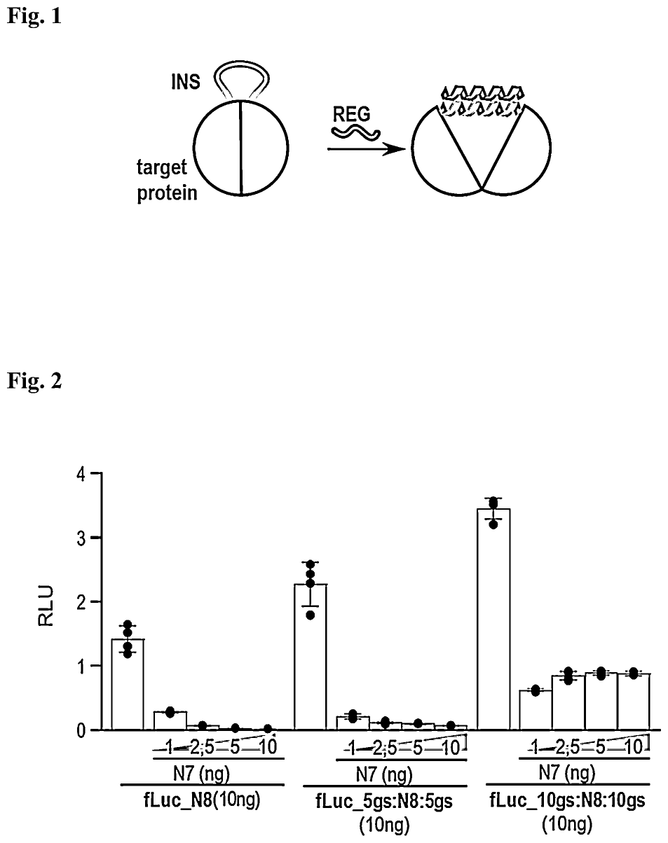

Peptides that serve as the inserted peptide, inhibitory peptide or regulatory peptide are selected from the set of designed coiled-coil dimers that typically comprise 20-50 amino acid residues. The peptides are in isolation weakly structured or nonstructured and only form a coiled-coil dimer in the presence of the partner peptide from the set of designed coiled-coil heterodimers (Drobnak et al., 2017; Gradisar & Jerala, 2011; Plaper et al., 2021) (FIG. 1). When inserted into the protein loop, the distance of protein termini at the insert are less than 2 nm, however when the coiled-coil dimer is formed the distance between the termini of the insertion site in the protein increases to 3-4 nm, corresponding to the length of the coiled-coil dimer, which results in the allosteric effect on the protein function. The site for introduction of an inserted peptide into the target protein is selected based on the known tertiary structure or a structural model of the target protein. Sites for insertion are preferentially selected from the loop sites that are hydrophilic and highly exposed to the solvent, that are typically more variable than the hydrophobic core of the protein and that are not directly part of the interaction or catalytic or other functional site of the selected protein. Additionally, the site for interaction is more variable, less conserved, and preferentially may have different lengths of the loop in homologues. While not directly part of the functional site, the insertion site should be near the functional site, typically less than 2 nm away in order to transduce the allosteric effect on protein function.

The insertion site is selected in a manner that insertion of a peptide maintains the function of the protein that we are interested in, such as the catalysis, dynamics or binding. According to the invention, typically several insertion sites can be functional for each protein.

In the present invention the appropriate target protein site is identified by testing the function of the target protein with an inserted peptide inserted at different, typically solvent-exposed, loop sites that do not participate directly in protein function. The position of the insertion within the selected protein is selected in such way that the target protein with an inserted peptide retains the function. In the presence of an interaction peptide, the target peptide forms a coiled-coil dimer with a peptide inserted into the target protein loop, which impairs the function of the selected protein.

The present invention also refers to the method of activation of a target protein function (ON switch), where the target protein is initially inhibited through a combination of an inserted peptide as described above with an additional C- or N-terminal fusion to the target protein of an inhibitory peptide that interacts weakly with an inserted peptide and forms a coiled-coil dimer with a inserted peptide within a target protein, wherein the inhibitory peptide is genetically fused to the target protein via a flexible peptide linker. Weaker affinity between the inserted peptide and an inhibitory peptide in this context means affinity in the micro- to millimolar concentration range, that is sufficient to form intramolecular coiled-coil dimer yet can be displaced by a stronger, binding regulatory peptide, with affinity in the nanomolar or lower range. Weaker affinity between the inserted peptide and inhibitory peptide also ensures that the inhibitory peptide dissociates if it is no longer covalently connected to the target protein, which can be accomplished by the proteolytic cleavage of the linker between the protein and inhibitory peptide.

Said target protein can be activated by the addition of a regulatory peptide that strongly interacts with an inhibitory peptide or through a protease that cleaves the linker between the target protein and the inhibitory peptide. If a regulatory peptide with high affinity for the inhibitory peptide is added the inserted peptide is outcompeted from the inhibitory peptide and the protein regains the activity.

The present invention can be further implemented to regulate the function of the target protein by a protease whose activity can be regulated through small molecules that can trigger reconstitution of a split protease or though proteases characteristic for biological processes, such as e.g. proteases specific for the cell type, physiological cell state or a microbial protease, for example viral protease present due to the infection.

For precise recognition or response more than one input signal may be required, that can more precisely specify the context in which a selected biological process should be activated. The presence of the first signal and the absence of the second signal or presence of both signals or presence of at least one of the two signals or other combinations of input signals define logic functions, which could be beneficial to precisely specify activation of the selected protein that may have a role in a physiological, therapeutic or biotechnological process. The invention describes construction of those logic functions that are achieved by combinations of insertions and fusions of interacting peptides and protease cleavage sites that can be combined to generate logic functions that combine two or more input signals that define under which conditions the selected protein is in active or inactive form. The proteins can also be proteases that are able to represent an input signal to the logic gates at higher level and generate more complex logic circuits.

This invention refers to activation or inhibition of the diverse biological function or biological activity such as catalytic activity or binding or other function of various target proteins such as, but not exclusively, enzymes, such as luciferase, hydrolases, kinases or proteases, proteins that constitute signaling pathways, such as protein kinases, signaling mediators that recruits other proteins to the signaling complex, further it refers to proteins which bind specific nucleic acid sequences or that act as transcriptional regulators, and proteins that can be detected and act as reporters, such as fluorescent proteins or luciferases and further it refers to other molecule-recognizing domains such as antibodies and their domains or to structural proteins.

The disclosed invention refers to functional proteins whose activity can be inhibited or activated by the addition or presence of a chemical or biological signal that affects the formation of the dimer involving the inserted peptide in the loop of the target protein.

The disclosed invention refers to the method of regulating the activity of proteins and said proteins and nucleic acids encoding them and cells producing said proteins.

In the particular embodiment, the disclosed invention refers to the activation or inhibition of the biological and biochemical function of proteins luciferase, beta-galactosidase, IRAK-1 kinase, MyD88, Lyn kinase, transcription activator-like effector and its implementation as DNA binding domains, Cas9 gRNA-dependent DNA nuclease or specific DNA binding domain, fluorescent proteins, antibody variable domain (Fv), whose activity can be regulated through interactions with a peptide that forms a coiled-coil heterodimer with a peptide inserted into the loop of the target proteins.

In the particular embodiments, the cells such as the mammalian or eukaryotic or bacterial cells exhibit the function of target proteins depending on the applied signal, which triggers formation or reverses formation of a coiled-coil dimer within the target protein loop that results in the modification of the biological properties or cells that produce the modified target proteins, which can be used for medical, biotechnological or other application of cell function.

Moreover, the present invention refers to nucleotide sequences comprising coding sequences for polypeptides described above according to the invention, optionally incorporated in a delivery vector such as a plasmid, a linear or circular nucleic acid or a virus or inserted into the genome of cells. In a further aspect, the present invention refers to a protein comprising the polypeptide according to the invention.

The invention refers to a method of regulating a function of a target protein by:
a. genetically inserting an insertion peptide with a length of 20 to 50 amino acid residues into the selected target protein at such a position of the target protein, preferably in a solvent exposed loop, separated from the site responsible for the protein function, that the function of the target protein is maintained
b. adding a regulatory polypeptide, which specifically interacts with said inserted peptide and forms a structured dimer and increases the distance between the termini of the inserted peptide and inhibits or inactivates the function of the target protein.

The invention refers to the method to allosterically regulate function of the target protein wherein the peptide is inserted into said target protein where the regulatory peptide pair to regulate function of the said protein are selected from the designed heterodimeric pairs comprising or consisting of the amino acid sequences as shown in SEQ ID NO: 121 to SEQ ID NO: 146.

The invention refers to the method of regulating a protein function, wherein:

An inserted peptide that is by itself not structured is inserted into the selected target protein at such position of the target protein, preferably into a solvent exposed loop, that the function of the protein is maintained.

Inhibitory peptides that can interact with the inserted peptide in the target protein according to a) are fused to the target protein via a nonstructured linker peptide, preferentially comprising 3 to 30 amino acid residues, more preferably 5 to 20 amino acid residues or 10 to 15 amino acid residues, wherein the inhibitory peptides can bind to the inserted peptide within the same molecule thereby inhibiting the function of said protein in the constitutively expressed state, wherein the function of the target protein can be regained by the addition of a regulatory peptide which binds tightly to the inhibitory peptide or by cleaving the linker between the inhibitory peptide and the target protein by a protease that has a recognition sequence within the nonstructured linker.

The invention refers to a method of allosteric regulation of a protein function based on insertion of a peptide into said target protein and fusion of an inhibitory protein to the target protein, wherein the activity of the target protein is regulated by the proteolytic cleavage by a protease that cleaves between the target protein and the inhibitory peptide, wherein the protease can be regulated by a small molecule or wherein a protease is used that is characteristic for a desired physiological process or is derived from pathogen, such as a virus, bacteria, fungi or a parasite.

The invention refers to a method of allosteric regulation of protein function by insertion of a peptide, where the function of the target protein is regulated by combination of input signals that affect the structure of the insert peptide which comprise logic functions to combine several chemical or biological signals.

The invention refers to proteins allosterically regulated by insertion of a peptide where the function of the target proteins can be regulated by the addition or release of a peptide that regulates the function of the target protein wherein the target protein is an enzyme or a DNA binding protein domain or a signaling protein.

The invention refers to proteins allosterically regulated by the peptide that interacts with the peptide inserted or fused to the target protein where the target protein is a firefly luciferase or MyD88 or IRAK1 or Lck kinase or beta-galactosidase or tobacco-etch virus protease or a Transcription-activator-like effector or Cas9 or antibody or its single chain variable domain (scFv) comprising or consisting of an amino acid sequence as shown in SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, or 120.

The invention refers to an allosterically regulated protein based on insertion of a peptide into a target protein wherein the target protein is an antibody or nanobody or its single chain variable domain (scFv) of SEQ ID NO: 116.

The invention refers to nucleic acids coding for proteins with sequences as shown in SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, or 145.

The invention refers to cells containing nucleic acids coding for allosterically regulated proteins based on insertion of a peptide into the target proteins where the properties of the cell are regulated by the proteins encoded by the nucleic acids and peptides interacting with them.

The inventions refer to a method of regulating the response of immune cells, preferentially T cells expressing chimeric antigen receptor (CAR), wherein the recognition of target cells is achieved by an allosterically regulated antibody domain, wherein the recognition of proteins on target cells can be regulated by the addition of a peptide that binds to the insert in the variable fragment of the antibody and affects its recognition of target cells through the allosteric effect.

The invention refers to proteins comprising or consisting of an amino acid sequence as shown in SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 144, or 146.

The examples described in further detail have no intention of limiting the scope of the invention and its applicability, but are merely intended to provide a better understanding of the invention and its applicability.

### EXAMPLES

### Example 1: Design and Preparation of DNA Constructs for the Demonstration of the Invention

DNA constructs were prepared using methods of molecular biology that are described in any molecular biology handbook and are known to experts. In order to prepare DNA constructs, the inventors used experimental techniques and methods such as: chemical synthesis of DNA with a defined polynucleotide sequence, DNA fragmentation with restriction enzymes, DNA amplification using polymerase chain reaction-PCR, PCR ligation, DNA concentration determination, agarose gel electrophoresis, purification of DNA fragments from agarose gels, ligation of DNA fragments into a vector, the Gibson assembly method, transformation of chemically competent cells E. coli DH5a, isolation of plasmid DNA with commercially available kits, screening and selection. DNA segments were characterized by restriction analysis and sequencing.

All plasmids, completed constructs and partial constructs were transformed into bacterium E. coli DH5alfa by chemical transformation. Plasmids for transfection into the cell line HEK293T were isolated using GeneJet Plasmid DNA Isolation Kit (InVitrogen).

### Results

All protein coding constructs have a Kozak sequence (GCCACC) before the coding region and were cloned into the pcDNA3 backbone vector for high-level expression in mammalian cells.

In the described examples, the target proteins are modified by an inserted peptide, introduced into the positions determined from the tertiary structures or molecular models of proteins which are located in solvent exposed loops, that are close to the active or functional site of the target protein, typically within 2-3 nm.

The DNA coding for the modified target proteins were in-frame inserted into vectors. All protein-coding DNA construct demonstrated as examples are listed in Table 1.

TABLE 1
Target proteins used for the demonstration of the invention and positions
within the amino acid sequences where the inserted peptides were inserted

| DNA | Amino | | Position of inserted |
|---|---|---|---|
| SEQ | acid | | peptide in |
| ID | SEQ | Description | target protein |
| 1 | 2 | Firefly luciferase (fLuc_N8) | K493-T494 |
| 3 | 4 | Firefly luciferase (fLuc_5gs:N8:5gs) | K493-T494 |
| 5 | 6 | Firefly luciferase (fLuc_10gs:N8:10gs) | K493-T494 |
| 7 | 8 | Firefly luciferase (nLuc:5gs:P7:5gs:cLuc:20gs:N8) | K493-T494 |
| 9 | 10 | nLuc_5gs:P7:5gs:cLuc:AU1:gs6:SbMVs:gs6:N8 | A logic function |
| 11 | 12 | N8:gs6:PPvs:gs6:nLuc:5gs:P7:5gs:cLuc | B logic function |
| 13 | 14 | nLuc:5gs:P7:5gs:cLuc:AU1:gs20:N8:SbMVs:P7A | NOT B logic function |
| 15 | 16 | N8:gs6:PPVs:gs6:nLuc:5gs:P7:5gs:cLuc:AU1:gs6:SbMVs:gs6:N8 | AND logic function |
| 17 | 18 | nLuc:5gs:P7:5gs:cLuc:AU1:gs:PPVs:gs:SbMVs:gs:N8 | OR logic function |
| 19 | 20 | N8:gs6:PPVs:gs6:nLuc:5gs:P7:5gs:cLucAU1:20gs:N8:gs6:SbMVs:gs6:P7A | A nimply B |
| 21 | 22 | nLuc:5gs:P7:5gs:cLuc:AU1:gs6:SbMVs:gs6:N8:6gs:PPVs:6gs:P7A | A imply B |
| 23 | 24 | nLuc:5gs:P7:5gs:cLucAU1:gs20:N8:PPVs:P7A | NOT A logic function |
| 25 | 26 | nLuc:5gs:P7:5gs:cLucAU1:gs6:PPVs:gs6:N8:6gs:SbMVs:6gs:P7A | B imply A |
| 27 | 28 | N8:gs6:SbMVs:gs6:nLuc:5gs:P7:5gs:cLucAU1:20gs:N8:gs6:PPVs:gs6:P7A | B nimply A |
| 29 | 30 | β -galactosidase | L25-N26 |
| 31 | 32 | β -galactosidase | A35-S36 |
| 33 | 34 | β -galactosidase | T45-D46 |
| 35 | 36 | β -galactosidase | L55-N56 |
| 37 | 38 | β -galactosidase | D223-D224 |
| 39 | 40 | β -galactosidase | A229-T230 |
| 41 | 42 | β -galactosidase | A239-V240 |
| 43 | 44 | β -galactosidase | D507-E508 |
| 45 | 46 | β -galactosidase | P513-A514 |
| 47 | 48 | β -galactosidase | D610-R611 |
| 49 | 50 | β -galactosidase | T799-R800 |
| 51 | 52 | β -galactosidase | S1000-P1001 |
| 53 | 54 | TEV protease | G27-H28 |
| 55 | 56 | TEV protease | L72-Q28 |
| 57 | 58 | TEV protease | I77-D78 |
| 59 | 60 | TEV protease | G79-R80 |
| 61 | 62 | TEV protease | K147-D148 |
| 63 | 64 | TEV protease | F172-T173 |
| 65 | 66 | TEV protease | T175-N176 |
| 67 | 68 | TEV protease | K184-N185 |
| 69 | 70 | TEV protease | G212-G213 |
| 71 | 72 | TEV protease (TEVp_I77_5gs:P7:5gs_gs:N8) | I77-D78 |
| 73 | 74 | Lck kinase | N266-G267 |
| 75 | 76 | Lck kinase | Q256-F257 |
| 77 | 78 | Lck kinase | G278-S279 |
| 79 | 80 | Lck kinase | D374-T375 |
| 81 | 82 | IRAK 1 | F212-G213 |
| 83 | 84 | IRAK 1 | G221-G222 |
| 85 | 86 | IRAK 1 | R232-G233 |
| 87 | 88 | IRAK1 | S281-G282 |
| 89 | 90 | MyD88 | T66-R67 |
| 91 | 92 | MyD88 | G80-R81 |
| 93 | 94 | MyD88 | N170-D171 |
| 95 | 96 | MyD88 | S209-E210 |
| 97 | 98 | MyD88 | S224-D225 |
| 99 | 100 | TALA (TALA_T496_N6_N8) | T496-P497 |
| 101 | 102 | TALA (N5_TALA_N6_N8) | T496-P497 |
| 103 | 104 | dCas9 | S55-G56 |
| 105 | 106 | dCas9 | D147-K148 |
| 107 | 108 | dCas9 | R535-K536 |
| 109 | 110 | dCas9 | G1104-F1105 |
| 111 | 112 | dCas9 | K1153-K1154 |
| 113 | 114 | dCas9 (N8:gs40_dCas:VPR_R535_5gs:P7:5gs) | R535-K536 |
| 115 | 126 | scFv | S196-K197 |
| 117 | 118 | ngGFP (ngGFP C143_P7) | C143 |
| 119 | 120 | ngGFP (ngGFP C143_P7_N8) | C143 |

5gs linker of 5 aminoacids in length consisting of serine and glycine
10gs linker of 10 aminoacids in length consisting of serine and glycine

TABLE 2
List of inserted peptides, regulatory peptides and inhibitory peptides

| DNA | AA | Peptide | |
|---|---|---|---|
| SEQ ID NO | SEQ ID NO | name | Amino acid sequence |
| 121 | 122 | N8 | YKIAALKAENAALEAKIAALKAEIAALEAGC |
| 123 | 124 | N7 | EIAALEAKNAALKAEIAALEAKIAALKAGY |
| 125 | 126 | P7A | EIAALEAKNAALKAEIAALEAKNAALKAGC |
| 127 | 128 | P8A | YGKIAALKAENAALEAKIAALKAENAALEA |
| 129 | 130 | P7 | EIQALEEKNAQLKQEIAALEEKNQALKYG |
| 131 | 132 | P7SN | EIQQLEEKNSQLKQEISQLEEKNQELKYG |
| 133 | 134 | P8 | SPEDKIAQLKEENQQLEQKIQALKEENAALEYG |
| 135 | 146 | N5 | GEIAALEAKIAALKAKNAALKAEIAALEA |
| 137 | 138 | N6 | KIAALKAEIAALEAENAALEAKIAALKAGY |
| 139 | 140 | P5A | YGENAALEAKIAALKAKNAALKAEIAALEA |
| 141 | 142 | P6A | KNAALKAEIAALEAENAALEAKIAALKA |
| 143 | 144 | P3 | SPEDEIQQLEEEIAQLEQKNAALKEKNQALKY |
| 145 | 146 | P4 | SPEDKIAQLKQKIQALKQENQQLEEENAALEY |

### Example 2: Design and Demonstration of Regulatory Peptide Mediated Inhibition of the Protein Activity

To demonstrate the design of inhibition of protein function by regulatory peptides, the inventors inserted the insertion peptide into the target protein into the position exposed to the solvent in the vicinity but not directly in the active or binding site of the target protein. The inserted peptide is selected from the set of designed pairs of coiled-coil heterodimers (Drobnak et al., 2017; Gradisar & Jerala, 2011; Plaper et al., 2021), where each individual peptide is in isolation intrinsically unstructured, however in the presence of the regulatory peptide, which is its designed binding partner, it adopts a helical secondary structure, and the inserted and regulatory peptides form a coiled-coil dimer (FIG. 1). The constructs for the modified target protein were expressed in the mammalian HEK293T cell line. When a regulatory peptide was co-expressed, either alone or in fusion with other proteins or peptide tags, it binds to the inserted peptide within the target protein, forming a coiled-coil dimer. This results in structuring of the inserted peptide and extension of the distance between the amino acid residues bordering the inserted site of the target protein. This locally disrupts the aid protein structure, triggering an allosteric effect on protein function. In case of firefly luciferase, the addition of a regulatory peptide leads to inactivation of the luciferase catalytic activity.

Plasmids encoding for the split firefly luciferase, were transiently transfected into the HEK293T cell line. A constitutively expressed Renilla luciferase (phRL-TK http://www.promega.com/vectors/prltk.txf) was used as control of transfection efficiency to quantitatively determine the effect on the firefly activity.

### Cell Culture, Transfection and Stimulation

Methods and techniques for cell line cultivation are well-known to experts in the field and are explained here only indicatively, with the intention of clarifying the example. Cell cultures of HEK293T cells were grown at 37° C. and 5% CO₂. For cultivation, DMEM medium with 10% FBS, containing all the necessary nutrients and growth factors was used. When the cell population reached a sufficient density, the cells were transferred to a new flask and/or diluted. For use in experiments, the number of cells was determined by the Countess automated cell counter (Invitrogen). A 96-well microtiter plate, suitable for growing cell cultures, was inoculated with 2*10⁴ cells per well 24 hours prior to transfection. Inoculated plates were incubated at 37° C. and 5% CO₂. At 30-90% confluence, they were transfected with a mixture of DNA and PEI (6 μl/500 ng DNA, stock concentration 0.324 mg/ml, pH 7.5) and further incubated at 37° C. and 5% CO₂. Cells were lysed 24-48 hours after transfection with passive lysis buffer (Promega) according to manufacturer's instructions. The inventors first measured firefly activity (Fluc), followed by renilla luciferase activity (Rluc). Renilla luciferase is expressing independently and presents the transfection efficiency; firefly luciferase is a direct output for our designed systems. The Fluc/Rluc ration (RLU; relative luciferase units) is thus the Fluc values normalized to transfection efficiency of each individual well.

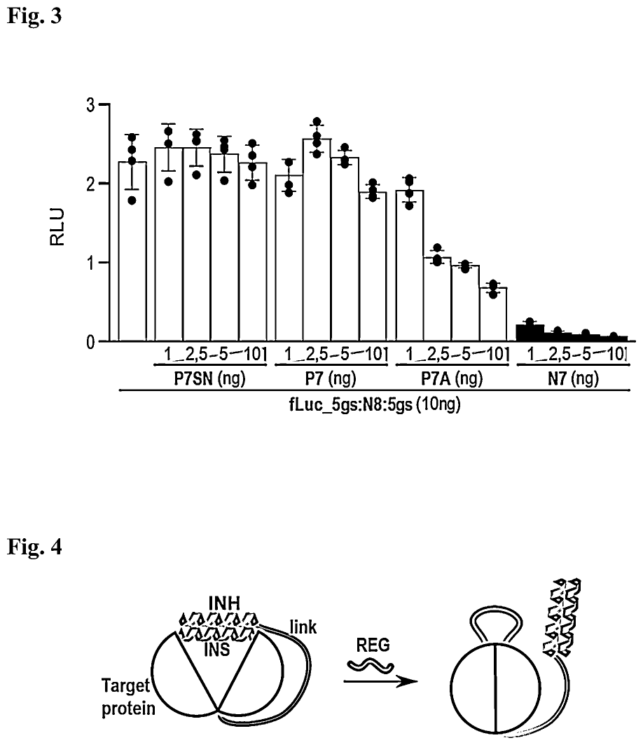

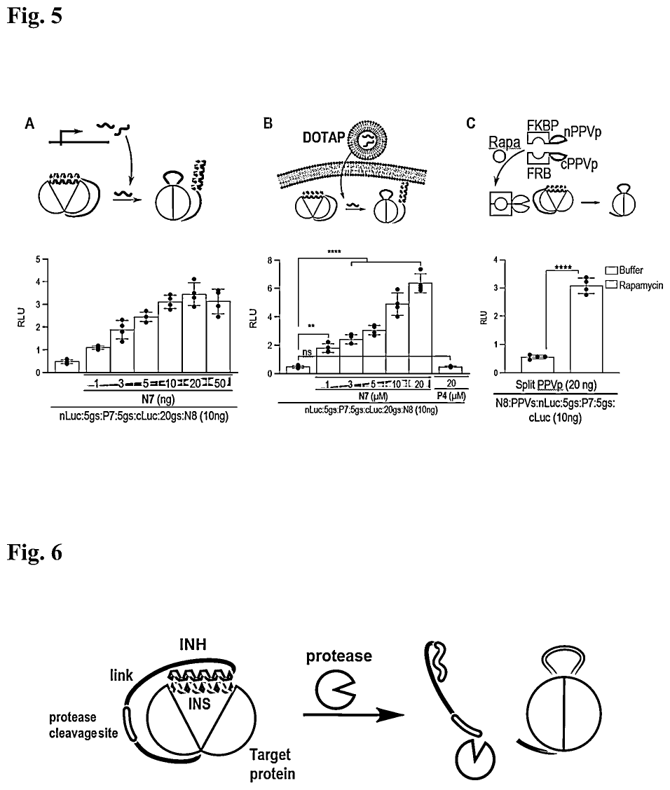

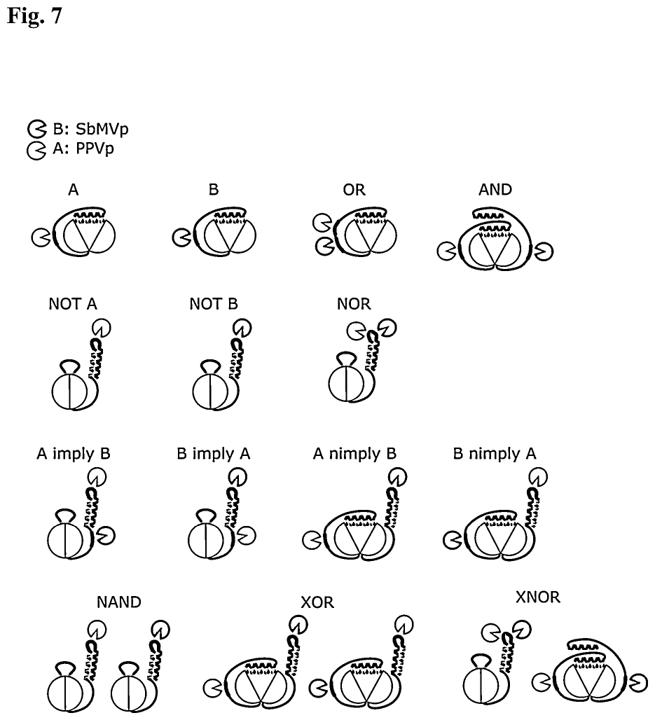

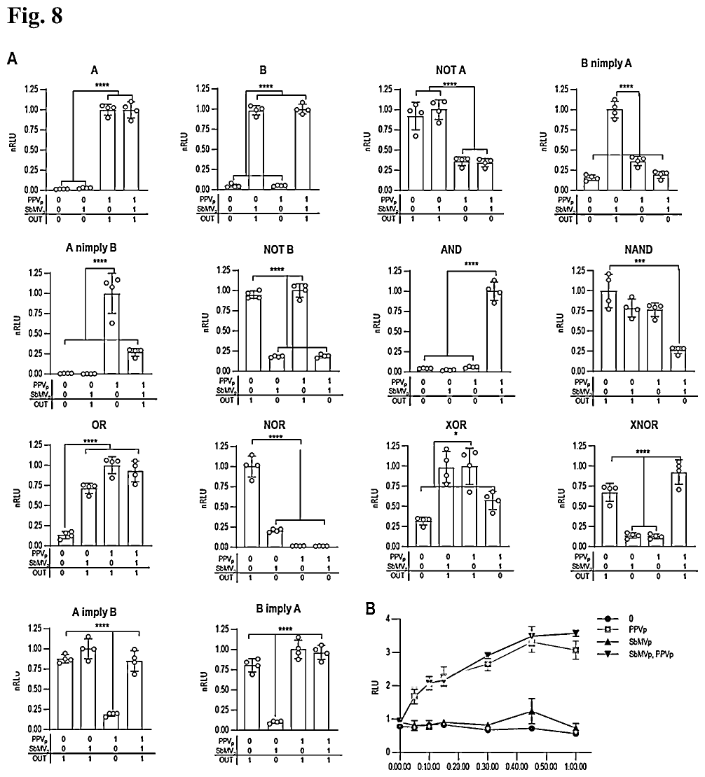

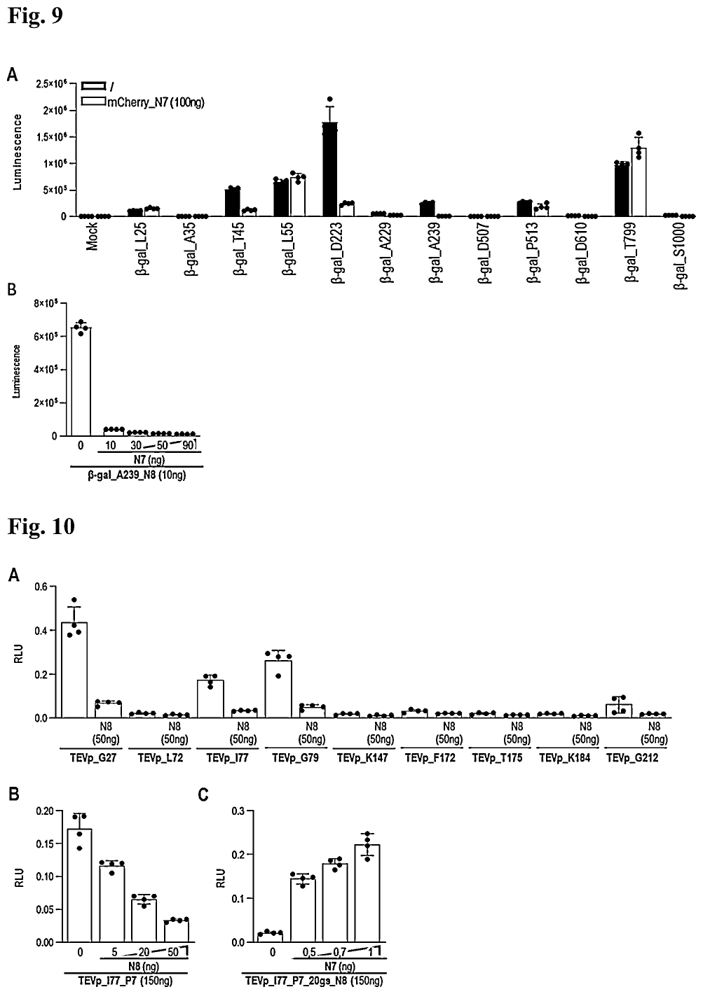

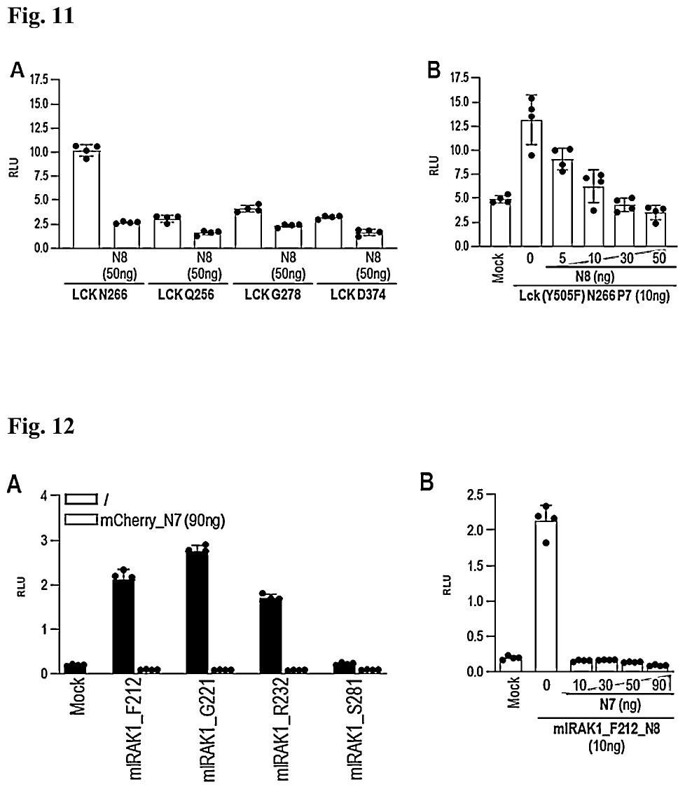

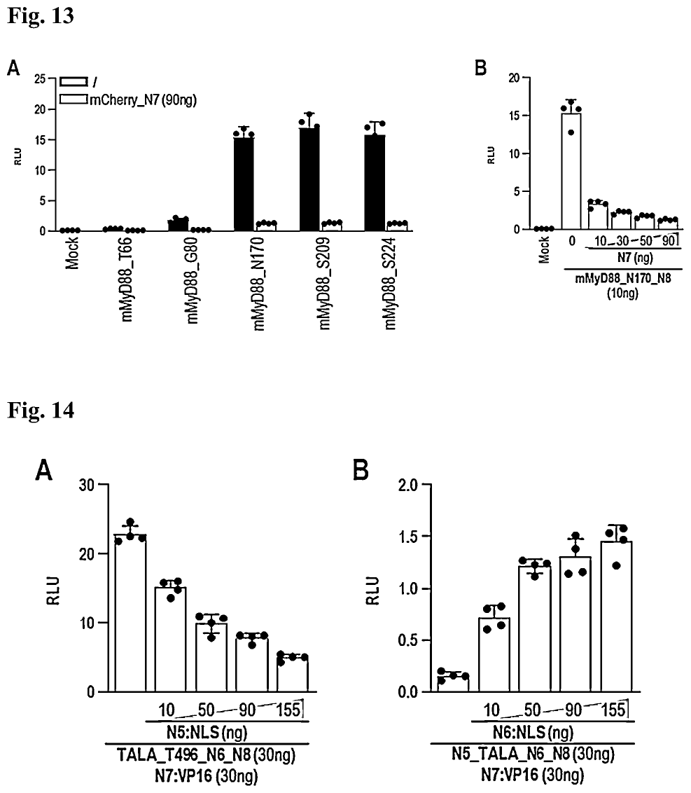

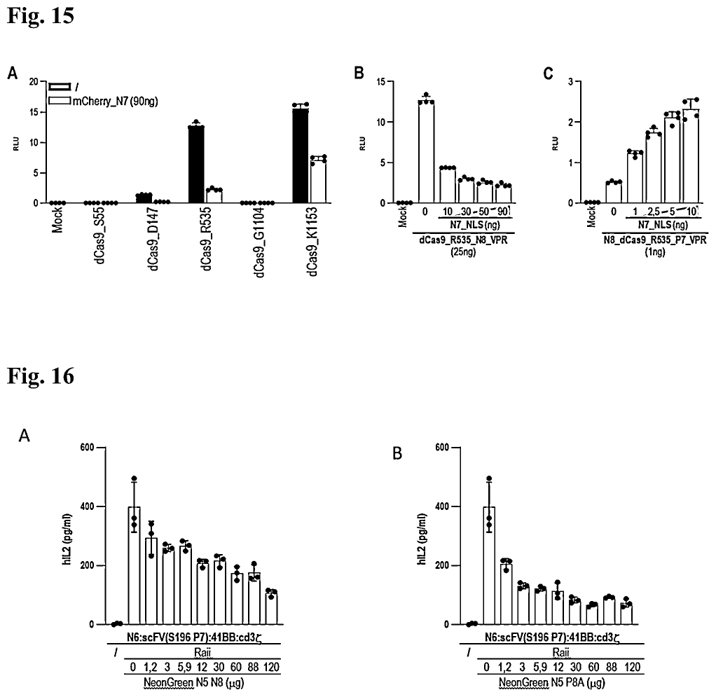

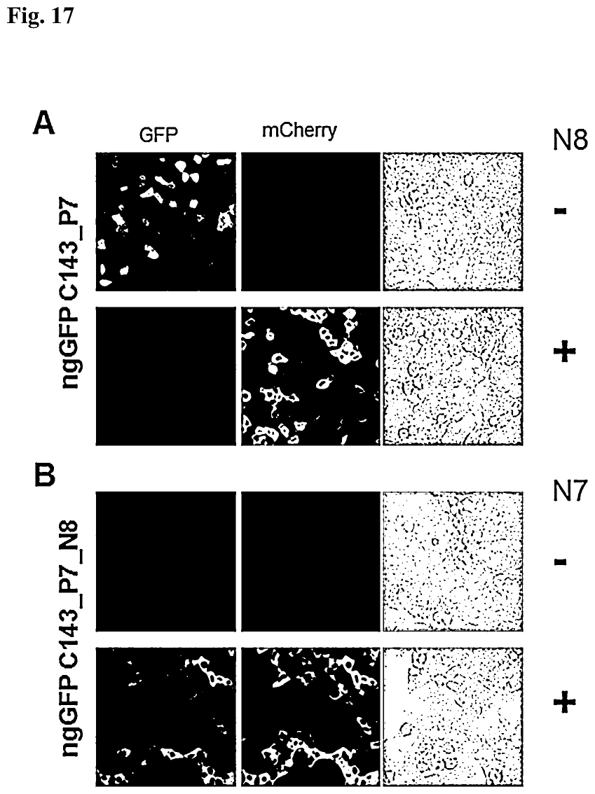

Results: The results shown in FIG. 2 demonstrate that insertion of a CC dimer-forming peptide N8 into an unstructured loop of a protein maintains its enzymatic function. Flexible peptide linkers allow for the target protein to retain its original tertiary structure conformation and function. Length of the linkers demonstrates that the linker peptide should preferably span between 0 and 10 amino acid residues composed of small hydrophilic amino acid residues such as glycine, serine, threonine. The absence of the linker decreases the activity of the target protein while 10 amino acid allows a more flexible region around the inserted peptide, which prevents the full inhibition of the function. The optimal length variant was shown to be 5 amino acid linkers on both sides of the inserted peptide. This variant retains high target protein activity in the absence of regulatory peptide and a strong inhibition in the presence of regulatory peptide N7.

In FIG. 3 the results support the dependency of peptide affinity on regulation of INSRTR target protein. Inserted peptide N8 can be paired with other regulatory peptides (P7SN, P7, P7A and N7), designed in a range of binding affinities. The affinity between the pairs of inserted and regulatory peptide has a strong effect on the function of target proteins. Higher affinity between inserted and regulatory peptide leads to better inhibition of activity of the target protein. It is therefore possible to predetermine the level of inhibition of the target protein by selection of the regulatory peptide with appropriate affinity to inserted peptide.

### Example 3: Demonstration of Activation of Protein Function of a Target Protein Using a Combination of Inserted, Inhibitory and Regulatory Peptides

Besides the inhibition of target protein activity or other function, it is sometimes desirable to have the protein in the inactive state until the signal for activation is applied. The inventors demonstrated the adaptation of inserted peptide for such an occasion. The inventors generated autoinhibited firefly luciferase reporter wherein the peptide was inserted peptide into loop and at the C-terminus of the luciferase the inventors extended the protein by 20 amino acids long flexible linker with a further appended sequence of the inhibitory peptide. Inhibitory peptide was selected to have a weaker affinity to the inserted peptide; however due to high local concentration the inhibitory peptide and inserted peptide as they are in the same polypeptide chain they form an intramolecular coiled-coil dimer. This disrupts the luciferase tertiary structure, maintaining the target protein in the inhibited state. Upon the addition of a regulatory protein, which was selected to have high affinity for the inhibitory peptide, the low affinity inhibitory inserted peptide coiled-coil is disassembled and a high affinity inhibitory peptide-regulatory peptide coiled-coil dimer forms. This leads to release of the inserted peptide, which loses its secondary structure and allows the target protein to attain its original tertiary structure and regain the functionality (FIG. 4).

Different ways for delivery of the regulatory peptide are possible. The inventors present three different strategies, although additional types of delivery known in the art are possible: co-expression regulated by the transcriptional regulation; synthetic peptide delivery into cells; externally regulated protease that cleaves the linker between the protein and inhibitory peptide, which leads to the inhibitory peptide release.

The plasmids encoding for INSRTR modified firefly luciferase, were transiently transfected into the HEK293T cell line. A constitutively expressed Renilla luciferase (phRL-TK http://www.promega.com/vectors/prltk.txf) was used as control of transfection efficiency.

The HEK293T cells were cultured and transfected as described above. For the regulatory peptide delivery with transfection reagent, 24 hours after transfection cells were stimulated with the mix of peptide with DOTAP transfection reagent. After 6 hours the media was removed and cells were lysed and measured as described above. External regulation of target protein activity was achieved with rapamycin stimulation 24 hours post transfection. Cells were lysed 24 hours post stimulation and measured as described above.

Results: As seen in FIG. 5 including an inserted peptide P7 and an inhibitory peptide N8 into the sequence of firefly luciferase substantially reduced its catalytic activity. Upon co-expression (FIG. 5a) or DOTAP mediated delivery (FIG. 5b) of regulatory peptide N7, firefly luciferase activity was regained corresponding to the amount of regulatory peptide present. Similar, by rapamycin stimulation (FIG. 5c) the inventors induced the reconstitution of split protease PPV which cleaved its catalytic site (PPVs) inserted into linker peptide between regulatory peptide N8 and N-terminal of firefly luciferase. Cleavage of the linker peptide resulted in dissociation of regulatory peptide N8 and reconstitution of firefly luciferase activity.

### Example 4: Demonstration of Performance of Logic Functions Based on a Combination of Proteases or Chemical Signals Affecting the Function of a Target Protein

For precise recognition or response more than one input signal may be required, that can more precisely specify the context in which a selected biological process should be activated. Presence of the first signal and the absence of the second signal, presence of both signals, presence of only one signal and other combinations define logic functions, which could be beneficial to specify activation of the selected protein. The invention describes those logic functions.

The HEK293T cells were cultured, transfected and stimulated as described above.

Results: The results shown in FIG. 8 demonstrate the construction of binary Boolean logic gates based on the invention of activating or inactivating the target proteins by inserting an interacting peptide and combining fusions of inhibitory peptides with cleavage sites for one or several proteases in combination with proteases whose activity can be regulated by other molecules (FIG. 6). Logic functions can be achieved through different proteins and combinations of proteins that together implement the desired logic function (FIG. 7).

In one embodiment of the invention, logic functions can be designed based on the combination of chemical signals that affect the function of a target protein. To this end, split PPV and SbMV protease were used that are regulated by addition of rapamycin or abscisic acid. We constructed firefly luciferase with P7 coiled-coil-forming segment inserted at position K490 and intramolecular fusion of the N8 CC segment at the C-terminal ends. This allows intramolecular binding of CC segments, forming a dimer, and resulted in constitutive inactivation of firefly luciferase. The cleavage site for the PPV protease was introduced into the flexible linker between the C-termini of the luciferase and the N8 coiled-coil forming segment. Addition of rapamycin restored the function of PPV protease which cleavage of the N8 coiled-coil, resulting in an ON switch. In the same manner, logic functions can be constructed for other proteins that are allosterically regulated by the formation of an extended structure in the loop of the target protein.

### Example 5: Regulation of the Activity of β-Galactosidase Through Insertion of a Coiled-Coil Forming Peptide

β-galactosidase is an exoglycosidase which hydrolyzes the β-glycosidic bond formed between a galactose and its organic moiety. It is a powerhouse of energy production by breaking down lactose to galactose and glucose and is therefore commonly used in food industry for production of lactose-free products. Amino acids chosen for insertion of the inserted peptide were L25, A35, T45, L55, D223, A229, A239, D507, P513, D610, T799 and S1000.

HEK293T cells were cultured and transfected as described in the second example. At the time point of 24-48 hours post transfection, activity of beta-galactosidase was assessed with β-gal reporter gene assay, chemiluminescent (Roche) by manufacturers' instructions. Luminescence produced as a result of a chemical reaction directly correlates with the amount of active beta-galactosidase. Luminescence was recorded with Luminescence plate reader Orion II.

Results: As seen in FIG. 9 results demonstrate successful implementation of INSRTR strategy to enzyme β-galactosidase. We constructed several variants by insertion of peptide N8 thought the length of β-galactosidase. Our aim was to obtain positions that are tolerable for change and retain β-galactosidase catalytic activity. Out of those, we determined which variants can be downregulated with co-transfection of 100 ng plasmid encoding for regulatory peptide N7. Three candidates, β-gal_T45, β-gal_D223 and β-gal_A239 proved to have such regulatory ability. Best inhibition was detected with β-gal_A239 variant, diminishing the β-galactosidase catalytic activity at very low amounts of co-transfected regulatory peptide N7.

### Example 6: Regulation of the Activity of Tobacco-Etch Virus Protease Through Insertion of a Coiled-Coil Forming Peptide

Tobacco-etch virus protease (TEVp) is a highly sequence-specific cysteine protease. In synthetic biology, it is frequently used for the controlled cleavage of fusion proteins in vitro and in vivo. Many proteases are inactive in innate state and regain their activity upon specific signal is applied. The inventors therefore present INSRTR method for both downregulation and upregulation of TEVp activity. Amino acids chosen for insertion of the inserted peptide were G27, L72, 177, G79, K147, F172, T175, K184, G212.

HEK293T cells were cultured, transfected and measured as described in the second example. Cyclic firefly luciferase reporter with TEVp cleavage site (cycLuc_tevs) was used to determine the activity of TEVp.

Results: Results in FIG. 10 demonstrate successful implementation of INSRTR strategy to TEV protease. We designed TEVp variants with inserted peptide P7 on various positions. Activity of TEVp INSRTR variants was determined in the absence and presence of co-transfected 50 ng of plasmid encoding regulatory peptide N8. Four variants TEVp_G27, TEVp_I77, TEVp_G79 and TEVp_G212 presented the desired downregulation in the presence of regulatory peptide N8. Best inhibition of TEVp activity was achieved with TEVp_I77 variant, dependent on the amount of regulatory peptide N8 (FIG. 10b). However, inhibition of protease has a limited use in synthetic biology so we implemented also inverted INSRTR strategy to activate the protein function. We designed inhibited version of TEVp_I77 by genetic fusion of inhibitory peptide N8 at the C-terminal of TEVp, connected via 20 amino acid long flexible linker. The induction of TEVp activity was achieved by co-transfection of regulatory peptide N7, which has a higher affinity for inhibitory peptide N8 (FIG. 10c).

### Example 7: Regulation of the Activity of Lck Kinase Through Insertion of a Coiled-Coil Forming Peptide

Lck kinase is a member of the SRC family of non-receptor tyrosine kinases. They act by phosphorylating tyrosine residues, thereby regulating the activity of proteins involved in intracellular signaling of T cells. Lck kinase is the first kinase in the TCR signaling pathway and its activity controls the entire orchestra of kinases/phosphatases. N266, Q256, G278, D374 were chosen as amino acids for the insertion of the peptide. A specific phosphorylation-based luciferase reporter was constructed by introducing the phosphopeptide binding domain (SH2) and Lck specific substrate (GPLDGSLYAKVKKKD) into the luciferase such that the engineered luciferase becomes inactive and regains activity only when Lck kinase is present. HEK293T cells were cultured, transfected and relative luciferase activity was measured as described in the second example.

Results: FIG. 11 presents the demonstration of successful implementation of INSRTR strategy to enzyme and signaling mediator Lck kinase. Insertion of the peptide P7 at the N266 position retained Lck catalytic activity. Co-expression of various amounts of plasmid encoding for regulatory N8 peptide resulted in significant, dose-dependent inhibition of Lck activity, indicating potential for regulation of kinases using INSRTR strategy.

### Example 8: Regulation of the Activity of IRAK-1 Kinase Through Insertion of a Coiled-Coil Forming Peptide

The interleukin-1 receptor (IL-1R) associated kinase 1 (mIRAK1) is a member of serine/threonine-protein kinase. It plays a critical role against foreign pathogens by initiating innate immune response. Amino acids chosen for insertion of the inserted peptide were F212, G221, R232, S281.

HEK293T cells were cultured, transfected and measured as described in the second example. Activity of mIRAK1 was assessed by detection of downstream signaling cascade protein NF-κB. Reporter plasmid included NF-κB binding site in front of minimal promotor for firefly luciferase.

Results: FIG. 12 presents the demonstration of successful implementation of INSRTR strategy to enzyme and signaling mediator mouse IRAK-1. Several insertion positions of peptide N8 proved to be tolerable for such modification, namely mIRAK-1_F212, mIRAK-1_G221 and mIRAK-1_R232. All three variants retained mIRAK-1 function in signaling cascade for initiation of immune response that could be regulated by the presence of regulatory peptide N7. The variant mIRAK-1_F212 was further tested for downregulation in a dose-dependent manner of regulatory peptide N7, proving strong inhibition even at low amounts of regulatory peptide.

### Example 9: Regulation of the Activity of MyD88 Through Insertion of a Coiled-Coil Forming Peptide

MyD88 is involved in the innate immune response as adaptor protein in Toll-like receptor and IL-1 receptor signaling pathway. Amino acids chosen for insertion of the inserted peptide were located in death-domain T66, G80; and in Toll-Interleukin receptor (TIR) domain N170, S209, S224.

HEK293T cells were cultured, transfected and measured as described in the previous example.

Results: Results in FIG. 13 demonstrate successful implementation of INSRTR strategy to signaling mediator mouse MyD88. Out of the chosen Insertion positions of N8 peptide insertions only positions in TIR domain proved to be able to retain mMyD88 function, namely mMyD88_N170, mMyD88_S209 and mMyD88_S224. Downregulation of designed mMyD88 INSRTR variants was achieved by co-transfection of regulatory peptide N7 with all three successful variants. Further analysis of mMyD88_N170 with various amounts of regulatory peptide N7 displayed strong inhibition of mMyD88 function even with low amounts of regulatory peptide N7.

### Example 10: Regulation of the Activity of Transcription Activator-Like Effectors Through Insertion of a Coiled-Coil Forming Peptide

Transcription activator-like effectors (TALEs) is a DNA binding protein that in fusion with VP16 activator domain acts as a transcription activator. Amino acids chosen for insertion of the inserted peptide were T496.

HEK293T cells were cultured, transfected and measured as described in the second example. Reporter plasmid with ten TALE binding sites in front of the minimal promoter for firefly luciferase expression was used to determine the activity of TALE.

Results: Results in FIG. 14 demonstrate successful implementation of INSRTR and inverted INSRTR strategy to transcription activator-like effector (TALE-A). Here the insertion of N6 peptide was constructed at position T496 in the DNA binding region of TALE-A protein. Additional C-terminal fusion of peptide N8 to TALE-A protein directed interaction with activator domain VP16 in fusion with peptide N7 resulting in a functional Transcriptional activator. Co-transfection of N5 in fusion with nuclear localization signal (NLS) lead to in N5-N6 CC formation, resulting in decreased TALE-A DNA binding ability and inhibition of transcription of reporter protein (FIG. 14a). This presents another possible regulation of transcriptional activators. However, activation of transcription is also a useful tool in synthetic biology, thus we implemented inverted INSRTR strategy to activate the TALE-A DNA binding protein function. We designed inhibited version of TALE-A by genetic fusion of inhibitory peptide N5 on the N-terminal of TALE-A_T496. Co-transfection with regulatory peptide N6 with NLS signal results in dose-dependent increase of functional TALE-A, leading the transcriptional increase of reporter protein (FIG. 14b).

### Example 11: Regulation of the Activity of Cas9 Through Insertion of a Coiled-Coil Forming Peptide

Nuclease-dead Cas9 (dCas9) is a catalytically inactive mutant of Cas9 protein. In synthetic biology it is mainly used as a DNA binding protein which is guided to a specific DNA site by guide RNA (gRNA). In fusion with activator domain like VP16 or VPR it acts as a transcriptional activator. Amino acids chosen for insertion of the inserted peptide were S55, D147, R535, G1104, K1153.

HEK293T cells were cultured, transfected and measured as described in the second example. Plasmid with gRNA was used to direct dCas9 to the TALE binding sites on the reporter plasmid in front of the minimal promoter for firefly luciferase expression was used to determine the activity of dCas9.

Results: As shown in FIG. 15 we demonstrated successful implementation of INSRTR and inverted INSRTR strategy to transcription regulator dCas9. For implementation of INSRTR strategy, dCas9 was genetically fused to VPR activator domain, providing transcriptional activator. Regulation of transcription was achieved by insertion positions of peptide N8 at various positions in dCas9 protein. Two variants dCas9_R535 and dCas9_K1153 presented high retention of transcriptional activation function, however dCas9_K1153 variant was only two-fold inhibited in the presence of high amount of regulatory peptide N7. Co-transfection of regulatory peptide N7 with dCas9_R535 variant resulted in significant dose-dependent inhibition of transcription activation (FIG. 15b). We also implemented inverted INSRTR strategy to activate the dCas9 dependent transcription. We designed inhibited version of dCas9_R535 with inserted P7 peptide and N-terminal fusion of inhibitory peptide N8. Co-transfection with regulatory peptide N7 with NLS signal resulted in activation of transcriptional activity and higher transcription of reporter protein (FIG. 15c).

### Example 12: Regulation of the Binding of Single Chain Antibody Variable Domain and Regulation of Anticancer Activity of CAR T Cells Through Insertion of a Coiled-Coil Forming Peptide

The second generation CD19 CAR-T, bearing FMC63 single-chain variable fragment, with 4-1BB and cd3ζ (costimulatory domain recognizes CD19 ligand expressed by normal B cells and B cell leukemias and lymphomas. Chimeric antigen receptor composed of the scFv domain, hinge, transmembrane domain, 4-1 BB and CD3ζ (domain endows T cells with specific B-cell recognition and killing ability. The ability to control the activity of CAR T cells is very important for therapy as the excessive activation of CAR T cells may lead to the adverse therapeutic effects, such as the cytokine storm or on the other hand T cell exhaustion, which may either harm the patient or impair the anticancer ability of CAR T cells. Therefore, introduction of the ability to reduce the affinity of scFv to cancer antigens, such as CD19 but also others could be used to improve the therapeutic efficiency. Amino acid position for insertion of the inserted peptide into the scFv antigen recognition domain was S196.

Jurkat cells were grown at 37° C. and 5% CO₂. For cultivation, RPMI 1650 medium with 10% FBS, containing all the necessary nutrients and growth factors was used. When the cell population reached a sufficient density, the cells were diluted. For use in experiments, the number of cells was determined by the Countess automated cell counter (Invitrogen). Jurkat cells were mixed with a plasmid (10 μg), electroporated using Neon transfections system according to manufacturer's instructions and seeded into 2 mL of fresh RPMI 1650 medium. The next day, 24 h post electroporation the cells were counted and seeded into 96 well plate with target Raji cells in the ratio Effector (Jurkat-CAR-T):Target (Raji)=10:1. Stimulation with target cells was terminated after 24 hours by removal of media. Media was used for ELISA detection of interleukin-2 (IL-2), produced as a result of Jurkat CAR-T cell activation. The amount of IL-2 directly corresponds to activity of scFv-CD19.

### ELISA

Elisa test was performed to determine secreted hIL2 from electroporated and stimulated Jurkat cells. High binding half-well plates (Greiner) were used. Human IL-2 was measured using standard ELISA assay according to manufacturer's protocol (hIL-2 ELISA Invitrogen 88-7025-88). In brief, plates were coated with primary antibody and incubated overnight (4° C.) Next day plates were washed with PBS+0.05% Tween20 using ELISA plate washer (Tecan). Next, plates were blocked for 1 h at RT with ELISA diluent (PBS+3% FBS) solution. Afterwards, plates were again washed. Then serial dilution of hIL2 standard and 1:2 diluted samples were added and incubated at RT for 2 h. Next, plates were washed and afterwards detection antibody was added. Plates were incubated 1 h at RT. Next plates were washed and HRP conjugated avidin was added and incubated for 30 min. After the addition of substrate (TMB solution) the reaction was stopped with 0.16 m sulfuric acid. The plates were read on a microplate reader at 450 nm, and again at 630 nm for correction by subtraction of the reading at 630 nm from that at 450 nm.

Results: FIG. 16 shows the successful implementation of the INSRTR strategy in regulating the binding of antigen to an antibody. The P7 peptide flanking the 5 GS flexible linker was introduced into the single-chain variable domain of anti-CD19 at position N195/S196. The chimeric antigen receptor consisting of the INSRTR modified scFv domain, the CD8 hinge and transmembrane domain, the 4-1 BB and the CD3ζ (domain was used to regulate anticancer activity using the INSRTR strategy. CAR-T inhibition was achieved by adding a protein carrying the N8 CC pair. Jurkat cells expressing INSRTR-modified CD19 CAR T were able to secrete hIL2 after antigen challenge, whereas we observed a concentration-dependent inhibition of hIL2 secretion from cells to which a protein bearing the N8 CC pair was added.

### Example 13: Regulation of the Activity of Green Fluorescent Protein ngGFP Through Insertion of a Coiled-Coil Forming Peptide

Green fluorescent protein ngGFP is frequently used as a reporter. The inventors therefore present INSRTR method for both downregulation and upregulation of ngGFP activity. Amino acid chosen for insertion of the inserted peptide was C143.

HEK293T cells were cultured and transfected as described in the second example. Activity of fluorescent protein ngGFP was measured with confocal microscopy.

Results: Results in FIG. 17 demonstrate successful implementation of INSRTR strategy to ngGFP. We designed ngGFP variants with inserted peptide P7. Activity of ngGFP INSRTR variant was determined in the absence and presence of co-transfected plasmid encoding regulatory peptide N8. Addition of N8 regulatory peptide resulted in inhibition of ngGFP (FIG. 17a). We designed inhibited version of ngGFP by genetic fusion of inhibitory peptide N8 at the C-terminal of ngGFP, connected via 20 amino acid long flexible linker. The induction of ngGFP activity was achieved by co-transfection of regulatory peptide N7, which has a higher affinity for inhibitory peptide N8 (FIG. 17b).

### REFERENCES

Dagliyan, O., Dokholyan, N. V., & Hahn, K. M. (2019). Engineering proteins for allosteric control by light or ligands. Nature Protocols, 14(6), 1863-1883. https://doi.org/10.1038/s41596-019-0165-3
Dagliyan, O., Karginov, A. V., Yagishita, S., Gale, M. E., Wang, H., Dermardirossian, C., Wells, C. M., Dokholyan, N. V., Kasai, H., & Hahn, K. M. (2017). Engineering Pak1 Allosteric Switches. ACS Synthetic Biology, 6(7), 1257-1262. https://doi.org/10.1021/acssynbio.6b00359
Dagliyan, O., Shirvanyants, D., Karginov, A. V., Ding, F., Fee, L., Chandrasekaran, S. N., Freisinger, C. M., Smolen, G. A., Huttenlocher, A., Hahn, K. M., & Dokholyan, N. V. (2013). Rational design of a ligand-controlled protein conformational switch. Proceedings of the National Academy of Sciences of the United States of America, 110(17), 6800-6804. https://doi.org/10.1073/pnas.1218319110
Drobnak, I., Gradišar, H., Ljubetič, A., Merljak, E., & Jerala, R. (2017). Modulation of Coiled-Coil Dimer Stability through Surface Residues while Preserving Pairing Specificity. Journal of the American Chemical Society, 139(24). https://doi.org/10.1021/jacs.7b01690
Fink, T., Lonzarič, J., Praznik, A., Plaper, T., Merljak, E., Leben, K., Jerala, N., Lebar, T., Strmšek, Ž, Lapenta, F., Benčina, M., & Jerala, R. (2019). Design of fast proteolysis-based signaling and logic circuits in mammalian cells. Nature Chemical Biology, 15(2), 115-122. https://doi.org/10.1038/s41589-018-0181-6
Gao, X. J., Chong, L. S., Kim, M. S., & Elowitz, M. B. (2018). Programmable protein circuits in living cells. Science, 361(6408), 1252-1258. https://doi.org/10.1126/science.aat5062
Gradišar, H., & Jerala, R. (2011). De novo design of orthogonal peptide pairs forming parallel coiled-coil heterodimers. Journal of Peptide Science, 17(2). https://doi.org/10.1002/psc.1331
Ha, J.-H. H., & Loh, S. N. (2012). Protein conformational switches: From nature to design. Chemistry—A European Journal, 18(26), 7984-7999. https://doi.org/10.1002/chem.201200348
Langan, R. A., Boyken, S. E., Ng, A. H., Samson, J. A., Dods, G., Westbrook, A. M., Nguyen, T. H., Lajoie, M. J., Chen, Z., Berger, S., Mulligan, V. K., Dueber, J. E., Novak, W. R. P., El-Samad, H., & Baker, D. (2019). De novo design of bioactive protein switches. Nature, 572(7768), 205-210. https://doi.org/10.1038/s41586-019-1432-8
Mitrea, D. M., Parsons, L. S., & Loh, S. N. (2010). Engineering an artificial zymogen by alternate frame protein folding. Proc. Natl. Acad. Sci. USA, 107(7), 2824-2829. https://doi.org/10.1073/pnas.0907668107
Ostermeier, M. (n.d.). Engineering allosteric protein switches by domain insertion. https://doi.org/10.1093/protein/gzi048
Plaper, T., Aupič, J., Dekleva, P., Lapenta, F., Keber, M. M., Jerala, R., & Benčina, M. (2021). Coiled-coil heterodimers with increased stability for cellular regulation and sensing SARS-CoV-2 spike protein-mediated cell fusion. Scientific Reports 2021, 11(1), 1-16. https://doi.org/10.1038/s41598-021-88315-3

1. A method of regulating a function of a target protein comprising:
a. genetically inserting an insertion peptide with a length of 20 to 50 amino acid residues into the selected target protein at such a position of said target protein, preferably in a solvent exposed loop, that the function of the target protein is maintained;
b. adding a regulatory polypeptide, which specifically interacts with said inserted peptide and forms a structured dimer, in particular a heterodimeric coiled-coil with a defined structure, wherein this formation of a dimer, preferentially a coiled-coil peptide dimer, increases the distance between the amino and carboxy termini of the inserted peptide and inhibits the function of the target protein.

2. The method according to claim 1 where the inserted peptide and regulatory peptide pairs are selected from the designed heterodimeric pairs comprising or consisting of the amino acid sequences as shown in SEQ ID NO: 121 to SEQID 146.

3. A method of regulating a function of a target protein, comprising:
a. genetically inserting an insertion peptide that is by itself not structured into the selected target protein at such a position of said target protein, preferably into a solvent-exposed loop, that the function of the target protein is maintained;
b. fusing one or two inhibitory peptide(s) that can interact with the inserted peptide to the target protein via a non-structured hydrophilic flexible linker peptide, preferentially comprising 3 to 30 amino acid residues, wherein the inhibitory peptide can bind to the inserted peptide within the same protein molecule, preferentially forming a coiled-coil dimer, thereby inhibiting the function of the target protein in a constitutively expressed state;
wherein the function of the target protein can be regained by adding a regulatory peptide which binds tightly to the inhibitory peptide or
by cleaving the linker between the inhibitory peptide and the target protein by a protease that has a recognition sequence included within the linker peptide.

4. The method according to claim 3, wherein the activity of the protease is regulated by a small molecule or wherein the protease is characteristic for a desired physiological process or provided by a pathogen, such as a virus, bacteria, fungi or a parasite.

5. The method according to claim 1, wherein the function of the target protein is regulated by a combination of input signals that affect the structure of the inserted peptide, which form logic functions to combine several chemical or biological signals as input signals to provide as an output the desired protein in an active or inactive form.

6. The method according to claim 1, wherein the target protein is an enzyme, a nucleic acid sequence binding protein domain, a signaling protein, or a protein binding to any other molecule or antibody or its single chain variable domain (scFv), in particular a protein comprising or consisting of an amino acid sequence as shown in SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, or 120.

7. The method according to claim 1, wherein the target protein is selected from a firefly luciferase, MyD88, IRAK1, Lck kinase, beta-galactosidase, tobacco-etch virus protease, a Transcription activator-like effector, Cas9, or an anti CD19 single chain variable domain, in particular a protein comprising or consisting of an amino acid sequence as shown in SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, or 120.

8. The method according to claim 1, wherein the target protein is an antibody, a nanobody, or a single chain variable domain (scFv) thereof, in particular a protein comprising or consisting of an amino acid sequence as shown in SEQ ID NO: 116, 118, or 120.

9. A protein constructed according to claim 1.

10. A nucleic acid coding for a protein according to claim 9, in particular a nucleic acid comprising or consisting of an amino acid sequence as shown in SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, or 145.

11. A cell containing the nucleic acid according to claim 10, wherein the properties of the cell are regulated by the protein encoded by said nucleic acid and peptides interacting with it.

12. A method of regulating binding or recognition of target proteins or other molecules either free or at the surface of cells by proteins according to claim 1.

13. A method of regulating response of immune cells, preferentially T cells, expressing chimeric antigen receptor (CAR), wherein the recognition of target cells is achieved by an antibody domain according to the claim 8, wherein the recognition of selected proteins on target cells can be regulated by the addition of a peptide that binds to the inserted peptide in the variable domain of the antibody or nanobody and affects its recognition of target cells through the allosteric effect.
